(19) United States
(12) Patent Application Publication
(10) Pub. No.: US 2026/0287597 A1
(43) Pub. Date: Sep. 24, 2026

(54) POSTURAL ORTHOSTATIC TACHYCARDIA SYNDROME AND CRTH2

(71) Applicant: Amerimmune, LLC, McLean, VA (US)

(72) Inventor: Oral ALPAN, McLean, VA (US)

(73) Assignee: Amerimmune, LLC, McLean, VA (US)

(21) Appl. No.: 19/690,695

(22) Filed: May 28, 2026

Related U.S. Application Data
(63) Continuation of application No. 17/440,620, filed on Mar. 17, 2020, now abandoned.
(60) Provisional application No. 62/819,745, filed on Mar. 18, 2019.

(51) Int. Cl. G01N 33/68; A61K 31/519; G01N 33/88

(52) U.S. Cl. CPC G01N 33/6893; A61K 31/519; G01N 33/88; G01N 2333/70514; G01N 2333/70517; G01N 2800/322

(57) ABSTRACT

T cell surface marker, CRTH2, is a target for treating Postural Orthostatic Tachycardia Syndrome, a hemodynamic abnormality. Targeting CRTH2 permits control of disease symptoms in POTS, for which no FDA approved treatment is currently available. Cell surface expression on certain T cell subsets characterizes the syndrome. Cell surface expression can be conveniently determined in plasma samples using flow cytometry.

### CROSS-REFERENCE TO RELATED APPLICATIONS

This application is a Continuation of U.S. patent application Ser. No. 17/440,620 filed Sep. 17, 2021, which is the U.S. National Stage of International Application No. PCT/US20/23080 filed Mar. 17, 2020, which claims benefit of priority to U.S. Provisional Patent Application No. 62/819,745 filed Mar. 18, 2019, the entire contents of which are incorporated herein by reference.

### TECHNICAL FIELD OF THE INVENTION

This invention is related to the area of Autonomic Nervous System malfunctions. In particular, it relates to Postural Orthostatic Tachycardia Syndrome.

### BACKGROUND OF THE INVENTION

Dysautonomia is a term used to describe several different medical conditions that cause a malfunction of the Autonomic Nervous System. The Autonomic Nervous System controls the “automatic” functions of the body, such as heart rate, blood pressure, digestion, dilation and constriction of the pupils of the eye, kidney function, and temperature control. Postural Orthostatic Tachycardia Syndrome (POTS) is a subcategory of dysautonomia that impacts 1 out of 100 teenagers and, including adult patients, a total of 1,000,000 to 3,000,000 Americans, causing lightheadedness, fainting, tachycardia, chest pains, shortness of breath, GI upset, shaking, exercise intolerance, temperature sensitivity and more. Currently, no FDA approved treatment is available for POTS. There is a continuing need in the art to characterize, diagnose, and treat Postural Orthostatic Tachycardia Syndrome.

### SUMMARY OF THE INVENTION

According to one aspect of the invention a method is provided for stratifying a Postural Orthostatic Tachycardia Syndrome (POTS) patient for appropriate treatment. A sample of the patient is tested for cells that express on their surfaces prostaglandin D2 receptor 2 (CRTH2). The cells are one or both of memory CD4⁺ T cells and memory CD8⁺ T cells. The amount (absolute count or percentage) of cells in the sample of the patient that express on their surfaces CRTH2 is compared to the amount (absolute count or percentage) of cells which express on their surfaces CRTH2 in one or more healthy controls. A sample with significantly more cells expressing CRTH2 on their surfaces than in the healthy controls is identified. The patient from whom the sample was collected is stratified as appropriate for treatment with an antagonist of CRTH2.

According to another aspect of the invention another method is provided for stratifying a Postural Orthostatic Tachycardia Syndrome (POTS) patient for appropriate treatment. Maximum heart rate gap in the POTS patient is determined. The patient is treated by administering an antagonist of CRTH2 when the maximum heart rate gap is greater than or equal to 30 beats per minute.

According to still another aspect of the invention another method is provided for stratifying a Postural Orthostatic Tachycardia Syndrome (POTS) patient for appropriate treatment. A patient sample is tested for one or both of memory CD4⁺ T cells and memory CD8⁺ T cells that express on their surfaces prostaglandin D2 receptor 2 (CRTH2) by contacting antibodies with T cells of the patients so that the antibodies bind to the surfaces of subsets of the T cells, and subjecting the T cells with antibodies bound to their surfaces to flow cytometry to identify populations of cells that have surface-bound antibodies. The amount of cells expressing CRTH2 on their surfaces in the patient sample is compared to the amount of cells expressing CRTH2 on their surfaces in one or more healthy controls. A patient sample with significantly more cells expressing CRTH2 on their surface in the patient sample than in the healthy controls is identified. The patient from whom the sample was collected is stratified as appropriate for treatment with an antagonist of CRTH2.

According to another aspect of the invention a method is provided for treating a Postural Orthostatic Tachycardia Syndrome (POTS) patient. A POTS patient that expresses significantly more prostaglandin D2 receptor 2 (CRTH2) on the surfaces of a population of its cells than is expressed in the population of cells of a group of healthy controls is treated by administering to the POTS patient an antagonist of CRTH2.

Another aspect of the invention is a method of treating a Postural Orthostatic Tachycardia Syndrome (POTS) patient. A POTS patient that has a maximum heart rate gap greater than or equal to 30 beats per minute is treated by administering an antagonist of prostaglandin D2 receptor 2 (CRTH2).

Yet another aspect of the invention is a method of treating a Postural Orthostatic Tachycardia Syndrome (POTS) patient. An antagonist of CRTH2 is administered to a patient that has significantly more cells expressing prostaglandin D2 receptor 2 (CRTH2) on their surfaces than one or more healthy control individuals have. The cells are one or both of memory CD4⁺ T cells and memory CD8⁺ T cells.

These and other embodiments, which will be apparent to those of skill in the art upon reading the specification, provide the art with new ways to treat patients with POTS.

### BRIEF DESCRIPTION OF THE DRAWINGS

FIG. 1 shows the data from 16 patients looking at CRTH2 staining on CD4 and CD 45RO cells and correlating with maximum heart rate gap measured by tilt table testing. The data show statistically significant correlation between the clinical (heart rate gap) and immune cell staining (CRTH2 expression on CD4 T cells.

FIG. 2 shows plots of the expression of CRTH2 on T cells in patients with POTS. Percentage of CD4 and CD8 memory cells (CD4RO and CD8RO) expressing CRTH2 on the cell surface. There is a significantly higher percentage of CRTH2 expressing cells in patients with POTS compared to controls (p<0.008 for CD4 T cells and p<0.001 for CD8 T cells).

FIG. 3 shows exemplary flow cytometry data of CD4 and CD8 T cell surface expression of CRTH2 (detected using antibody to CD294) in a sample from a control, healthy individual.

FIG. 4 shows exemplary flow cytometry data of CD4 and CD8 T cell surface expression of CRTH2 (detected using antibody to CD294) in a sample from a patient with POTS. The CRTH2 expression can be seen in both memory and naïve CD4 and CD8T cells.

### DETAILED DESCRIPTION OF THE INVENTION

The inventors have developed assays for characterizing, diagnosing, and treating patients with Postural Orthostatic Tachycardia Syndrome (POTS). The assays permit the treatment of patients that overexpress prostaglandin D2 receptor 2 (CRTH2) with a therapeutic agent that targets and antagonizes CRTH2. Several such agents exist and can be used in treating POTS patients. These include, without limitation, fevipiprant, setipiprant, and CT-133. Additionally, the assays permit the stratifying of POTS patients into those for whom such antagonism will be useful or not useful.

In one aspect of the invention blood cells of the patient are analyzed to detect the expression of CRTH2 in particular subsets of T cells. In another aspect, patients are tested for a particular physiological symptom of POTS to determine appropriateness of treatment. Heart rate gap can be tested by detecting an abnormal heart rate increase upon standing. This can also be tested using a tilt table. Other symptoms of POTS may also be tested, including, but limited to, a drop in blood pressure upon standing, hypovolemia, high levels of plasma norepinephrine while standing, small fiber neuropathy.

Measurement of particular T cell subsets and cell surface expression of CRTH2 can be conveniently accomplished using flow cytometry. This assay employs antibodies that are specific for cell surface proteins. The antibodies are typically conjugated to fluorescent moieties that are detected by a flow cytometer.

Antagonists of CRTH2 are used to treat allergies and asthma. The antagonists can be used with similar dosing and regimens for treating POTS. Suitable antagonists which may be used include, without limitation, fevipiprant (2-(2-methyl-1-{[4-(methylsulfonyl)-2-(trifluoromethyl)phenyl]methyl}-1H-pyrrolo[2,3-b]pyridine-3-yl)acetic acid); setipiprant (2-[8-fluoro-2-(naphthalene-1-carbonyl)-3,4-dihydro-1H-pyrido[4,3-b]indol-5-yl]acetic acid); and CT-133 (C20H19FN3NaO4S).

In general, antibodies are a very convenient and sensitive way to detect and distinguish cells that differ in their expressed cell surface molecules. Antibodies can be used which are specific for CRTH2, CD4, and CD8, for example. Antibody to CD3 can be used to identify both CD4 and CD8 T cells. If naïve and memory cells are to be distinguished, antibodies can be used to accomplish that as well. For example naïve T cells express CD45RA whereas memory T cells express CD45RO. Additional means for distinguishing cells may involve gating with the flow cytometer. It is beneficial if each of the antibodies used in an assay is differentially fluorescently-labeled.

Samples which may be tested from patients and healthy controls may be any which contain T cells. Conveniently these can be obtained from blood, plasma or serum. A tissue source of T cells may also be used. Such tissues include, for example, skin, lung, and gastrointestinal tract.

In order to determine whether treatment with an antagonist of CRTH2 is appropriate, one may determine if the Postural Orthostatic Tachycardia Syndrome (POTS) patient has a maximum heart rate gap greater than or equal to 30 beats per minute. If the patient is 12 to 19 years of age, one would typically look for a maximum heart rate gap that is greater than or equal to 40 beats per minute before treating. Optimally one uses a tilt table to make this determination, but if such a device is not available, one can measure the difference in heart rate between laying down and standing (in the first 2, 5, and 10 minutes of rising from a supine position). One can test, in addition to heart rate, blood pressure. Some of the POTS patients will not exhibit orthostatic hypotension (>20 mm Hg drop in systolic blood pressure), and these patients are suitable for treatment as well.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

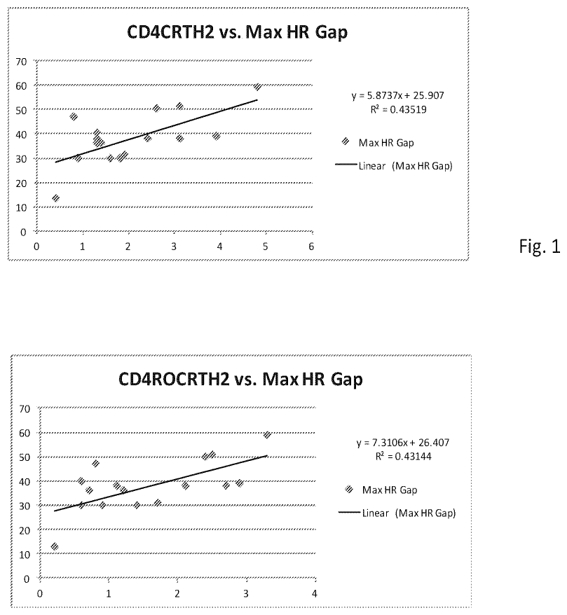

We compared hemodynamic parameters of 20 patients with POTS (M:F=1:4, age 16-34 y) to T cell surface CRTH2. There was a significant correlation (p<0.005) between memory CD4T cell CRTH2 expression and maximum heart rate gap, measured by the difference between supine and prone heart rate, a hallmark for POTS. Results are shown in FIG. 1.

There were no correlations between CRTH2 expression on CD8T cells or with other clinical parameters such as cardiac stroke index, maximum temperature difference or transcranial doppler mean cerebral blood flow velocity.

### EXAMPLE 2

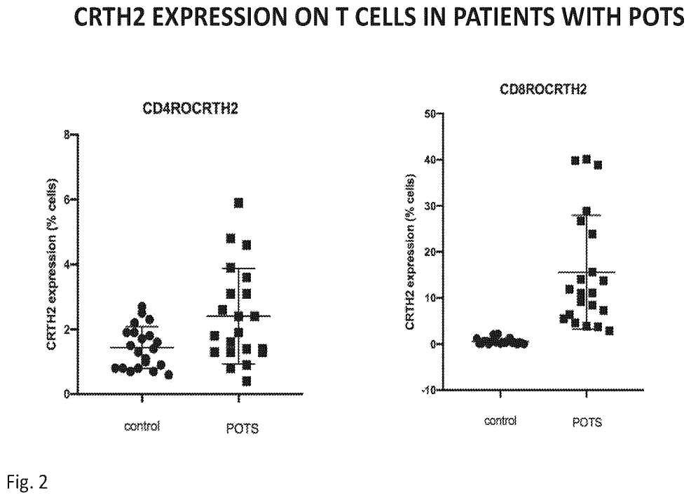

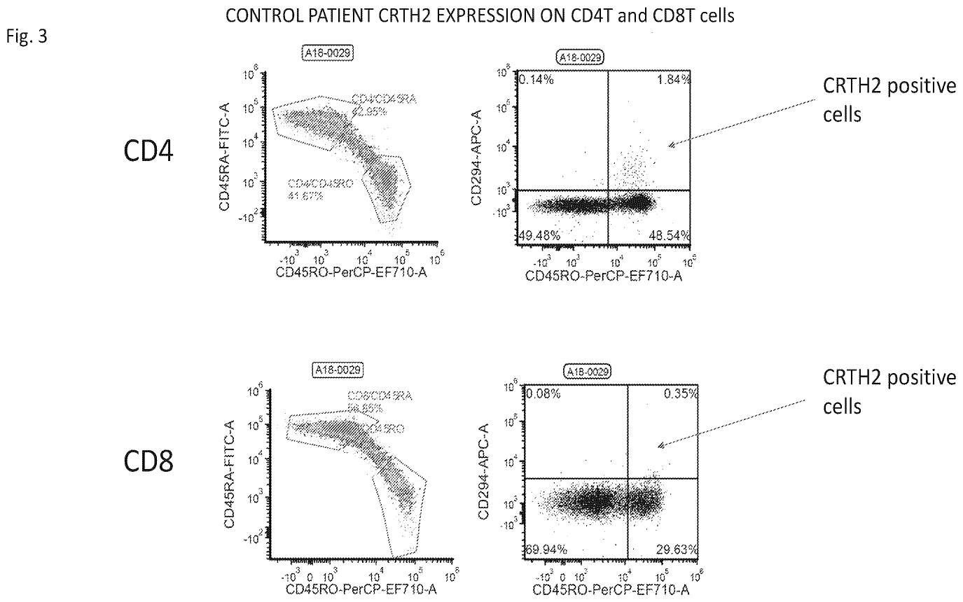

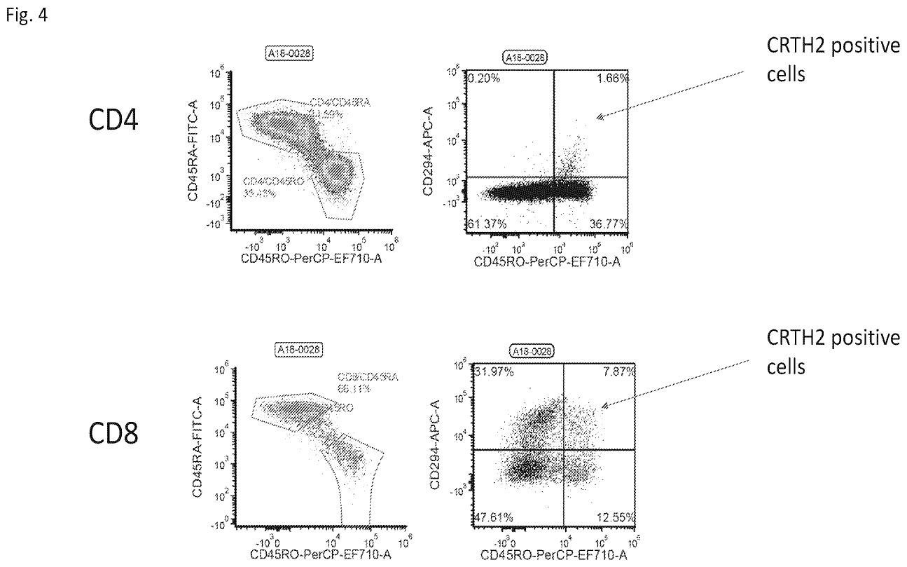

Overall CRTH2 expression in POTS patients (n=60) were significantly higher (7.8% and 2.1% respectively) in CD8CD45RO T cells (p<0.001) and CD4CD45RO T cells (p<0.005) compared to healthy age matched controls (0.9% and 0.4% respectively). See FIGS. 2-4.

### EXAMPLE 3

Four-color and three-color panels were used to detect cells expressing various relevant surface antigens. These included CD45RO FITC, CD45RA PE, CD8 PerCP, CD3 APC, CD4 PerCP, CD45RA FITC, CD45RO PerCP (eF710), CD3 PE (Cy 7), CD294 APC, CD4 (AF700), CD8a APC (eF780), and CD45 (eF506). These include the fluors Fluorescein isothiocyanate (FITC), R-phycoerythrin, Peridinin-chlorophyll protein, Allophycocyanin, Alexa Fluor® 700, and APC eFluor 780. The fluors were conjugated to antibodies that specifically bind to the named cell surface antigen.

In order to identify CD4⁺CD294⁺CD45RO⁺ and CD8⁺CD294⁺CD45RO⁺ T cell populations we:
1) Gated on Lymphocytes;
2) Gated on CD 45 to increase the purity of the lymphocyte population;
3) Used Forward scatter-Area vs Height to isolate singlets;
4) Gated on CD45+ Singlets that are CD3+;
5) Of the CD3+ Cells, separated and gated on CD4 and CD8;
6) Of the CD4+ and CD8+ positive population, we used expression of CD45RO and CD45RA to identify the memory and naive populations, respectively;
7) Then, of the CD8+ population, we used CD45RO and CD294 and determined the percent CD294+CD45RO+ cells. We also repeated the gating structure looking at CD4+ populations.

1.-38. (canceled)

39. A method of treating Postural Orthostatic Tachycardia Syndrome (POTS) in a patient, comprising:
testing a sample of the patient for cells that express on their surfaces prostaglandin D2 receptor 2 (CRTH2), wherein the cells are one or both of memory CD4+ T cells and memory CD8+ T cells;
comparing an amount of cells that express on their surfaces CRTH2 in the sample of the patient to an amount of cells that express on their surfaces CRTH2 in one or more healthy controls;
identifying the patient as having a sample with significantly more cells expressing CRTH2 on their surfaces than in the healthy controls; and
administering to the patient an antagonist of CRTH2.

40. The method of claim 39, wherein surface expression of CD4, CD8, or both CD4 and CD8 is separately assessed.

41. The method of claim 39, wherein antibodies are used to assess expression of one or more of CRTH2, CD4, and CD8.

42. The method of claim 39, wherein one or both of memory CD4+ T cells and memory CD8+ T cells are assessed using an antibody to CD45RO.

43. The method of claim 39, wherein an antibody to CD3 is used to identify one or both of CD4 T cells and CD8 T cells.

44. The method of claim 39, wherein flow cytometry is used to assess surface expression of T cell markers.

45. The method of claim 39, wherein testing the sample comprises contacting antibodies with T cells of the patient so that the antibodies bind to surfaces of subsets of the T cells, and
subjecting the T cells with antibodies bound to their surfaces to flow cytometry to identify populations of cells that have surface-bound antibodies.

46. The method of claim 39, wherein the antagonist of CRTH2 is selected from the group consisting of fevipiprant, setipiprant, and CT-133.

47. The method of claim 39, wherein the antibody is fluorescent-labeled.

48. A method of treating Postural Orthostatic Tachycardia Syndrome (POTS) in a patient, comprising:
determining that the patient has a maximum heart rate gap greater than or equal to 30 beats per minute; and
administering to the patient an antagonist of CRTH2.

49. The method of claim 48, wherein the patient is 12 to 19 years of age and has a maximum heart rate gap greater than or equal to 40 beats per minute.

50. The method of claim 48, further comprising testing for and determining that the patient does not exhibit orthostatic hypotension (>20 mm Hg drop in systolic blood pressure).

51. The method of claim 48, wherein the antagonist of CRTH2 is selected from the group consisting of fevipiprant, setipiprant, and CT-133.

52. A method of treating Postural Orthostatic Tachycardia Syndrome (POTS) in a patient, comprising:
administering to a POTS patient an antagonist of CRTH2,
wherein the POTS patient expresses significantly more prostaglandin D2 receptor 2 (CRTH2) on the surfaces of a population of its cells than is expressed in the population of cells of a group of healthy controls.

53. The method of claim 52, wherein the population of cells comprises one or more of CD4 cells, CD8 cells, memory CD4+ T cells, and memory CD8+ T cells.

54. The method of claim 52, wherein the antagonist of CRTH2 is selected from the group consisting of fevipiprant, setipiprant, and CT-133.

55. A method of treating Postural Orthostatic Tachycardia Syndrome (POTS) in a patient, comprising:
administering an antagonist of CRTH2 to a patient that has significantly more cells expressing prostaglandin D2 receptor 2 (CRTH2) on their surfaces than one or more healthy control individuals have,
wherein the cells comprise one or both of memory CD4+ T cells and memory CD8+ T cells.

56. The method of claim 55, wherein the cells comprise CD4 cells, CD8 cells, memory CD4+ T cells, memory CD8+ T cells, or a combination thereof.

57. The method of claim 55, wherein the antagonist of CRTH2 is selected from the group consisting of fevipiprant, setipiprant, and CT-133.
